(19) United States
(12) Patent Application Publication
(10) Pub. No.: US 2026/0363613 A1
(43) Pub. Date: Nov. 19, 2026

(54) TRANSESOPHAGEAL PACING AND DEFIBRILLATION APPARATUS

(71) Applicants: Bandula Wijay, Friendswood, TX (US); Nandhika Wijay, Houston, TX (US)

(72) Inventors: Bandula Wijay, Friendswood, TX (US); Nandhika Wijay, Houston, TX (US)

(21) Appl. No.: 19/262,936

(22) Filed: Jul. 8, 2025

Related U.S. Application Data
(62) Division of application No. 19/212,341, filed on May 19, 2025.

(51) Int. Cl. A61B 5/00; A61B 5/282; A61B 5/285; A61B 5/364; A61M 25/00; A61M 25/01; A61M 25/06; A61N 1/05; A61N 1/362; A61N 1/365; A61N 1/37; A61N 1/39; G16H 50/20

(52) U.S. Cl. CPC A61B 5/4836; A61B 5/282; A61B 5/285; A61B 5/364; A61B 5/6852; A61B 5/687; A61M 25/0026; A61M 25/0045; A61M 25/005; A61M 25/0068; A61M 25/0102; A61M 25/0662; A61N 1/0517; A61N 1/3621; A61N 1/365; A61N 1/3702; A61N 1/3925; A61N 1/395; A61N 1/39622; A61N 1/3968; G16H 50/20; A61B 2560/0468; A61M 2025/0037; A61M 2025/0046; A61M 2202/0007; A61M 2202/04; A61M 2205/0216; A61M 2205/0222; A61M 2205/0238; A61M 2205/3303; A61M 2205/3327; A61M 2205/3606; A61M 2205/50; A61M 2210/105; A61M 2230/04

(57) ABSTRACT

An esophageal cardiac pacing and defibrillation apparatus is disclosed. The apparatus has an elongated flexible shaft having a proximal end outside of the esophagus and a distal end in the esophagus near to the heart. The shaft includes a flexible distal end and a stiffening element that terminates before said distal end. This allows for ease of insertion and once inserted accurate navigation. An elongated pacing module is located on the outside of the shaft near its distal end. The cross section increases the length of contact with the esophageal wall optimizing the transfer of pacing energy. The pacing module is configured to measure heartbeat parameters and deliver regulating energy to the heart, and includes at least one pacing element on the outer surface of the pacing module, which can impart electric energy to the wall of the esophagus near to the heart or receive signals from the heart.

### FIELD

The present disclosure generally relates to devices and methods used for cardiac pacing and defibrillation. In particular, a method and apparatus for delivering electrical stimulation for cardiac and neuromuscular applications is disclosed.

### BACKGROUND OF THE DISCLOSURE

Cardiac arrhythmia is caused by irregularities in the electric signals that stimulate the contraction and expansion of the heart. Tachycardia, where the heart beats beyond the rate of the normal sinus rhythm, and bradycardia, where the heart beats below the normal rhythm are generally how an arrhythmia presents. The symptoms often include lightheadedness, shortness of breath and a decreased level of consciousness. These conditions and symptoms can increase the risk of stroke, leading to heart failure, or heart attack.

In some instances, cardiac arrhythmia does not present symptoms and goes untreated. When symptoms do present or the arrhythmia is otherwise discovered various treatments are available Antiarrhythmic drugs are often prescribed to patients. These drugs regulate the heartbeat by modifying the electric signals in the heart. There are multiple types or classes of these drugs that utilize various vehicles to regulate the electrical impulses of the heart. For example, Sodium Channel Blockers slow down electrical conduction while drugs classified as Beta-Blockers slow down the impulse conduction in the Sinoatrial (SA) Node and the Atrioventricular (AV) Node.

While therapeutics can be effective, they present limitations and can necessitate alternative solutions or require augmentation. Many antiarrhythmic drugs present a narrow therapeutic window making dosing a challenge. Another drawback is that some patients cannot tolerate the side effects of the drugs. Patients experiencing cardiac arrhythmia frequently present with other cardiac conditions requiring other drugs for treatment. The drugs taken to regulate the heartbeat can also adversely interact with these drugs. In some patients, antiarrhythmic drugs lack efficacy.

Arrythmia can be life threatening in which case therapeutics are not effective to regulate the heart. Instead, the delivery of a strong electric pulse or defibrillation is required. This delivers a shock to reset the heart's rhythm when it's beating too fast or erratically, potentially leading to cardiac arrest. For slow or irregular heartbeats that do not present conditions such as V-fib, pacing devices are utilized to regulate the heartbeat. In general, pacing is accomplished by connecting a regulated source of electricity to a target area proximally to the heart. The leads deliver electric pulses that regulate the heart rhythm. Pacing is generally classified as temporary or permanent.

Permanent pacing is utilized as a long-term solution and is accomplished by use of cardioverter pacemaker. This device is implanted using invasive techniques to place the device in close proximity to the heart's conduction system. Temporary pacing is utilized to stabilize the heart rhythm until the underlying condition is treated. Several temporary methods for pacing are utilized. For example, during transcutaneous pacing, pads are placed on the chest and back to deliver electrical impulses to the heart. This method is useful in emergency situations and when other methods are not immediately available. In epicardial pacing wires are placed directly on the outside surface of the heart during open-heart surgery. This method is commonly used during and after surgery and for temporary pacing.

Yet another temporary pacing technique utilizes transvenous pacing. This involves inserting a pacing lead through a vein, typically in the neck or chest, and into the heart. It is often used for longer-term temporary pacing or as a steppingstone to permanent pacing. Traditional transvenous pacemakers and implantable cardioverter defibrillators (ICDs) have been extensively studied and used to maintain cardiac rhythm in patients with conduction abnormalities. However, these devices require invasive implantation procedures and carry risks such as infection, lead displacement, and vascular complications.

Transesophageal pacing is a non-invasive alternative, particularly in electrophysiological studies, stress testing, and temporary cardiac pacing. Esophageal pacing utilizes the anatomical proximity of the esophagus to the left atrium and posterior aspects of the heart. This allows electrical stimulation to be applied with relative ease. Existing esophageal pacing techniques primarily involve the use of esophageal catheters inserted through the mouth or nose and positioned adjacent to the heart. The catheter houses the electrodes used for delivering the eclectic signals. These methods have been used for atrial pacing, vagal nerve stimulation, and certain diagnostic procedures.

Current esophageal pacing systems exhibit several limitations. It is important to locate the electrodes in the optimal position to ensure efficient pacing. The current designs make placement of electrodes challenging. In some instances, the placement results in poor electrode contact with the esophageal wall, leading to inconsistent pacing thresholds. Additionally, the placement of the electrodes may not always align optimally with the atrium or ventricle, leading to inefficient pacing and the inability of such devices to defibrillate the heart. Due to suboptimal electrode placement, higher energy levels are often required to compensate. This increases patient discomfort and the risk of esophageal irritation.

In many systems the catheter carrying the electrodes lacks integrated real-time electrocardiographic (EKG) feedback and the ability to measure other parameters such as oximetry. This can lead to inadequate control over pacing parameters, limiting adaptability to individual patient needs. This lack of information makes it difficult to dynamically optimize pacing settings. Where catheters are capable of multi-functional roles such as EKG monitoring, oximetry, temperature, and pacing, the catheter design is bulky to accommodate the multiple functions. Ideally, pacing should be accomplished with minimum disturbance of the esophagus. The bulky design of current systems leads to increased risk of esophageal irritation and injury. It is also difficult to access the esophagus for placement when the catheter is bulky.

What is needed is an esophageal pacing system that allows for optimized electrode placement such that defibrillation is also possible and a system that utilizes materials improving energy conduction and reducing stimulation thresholds. An optimal system will combine pacing with monitoring of another parameter and feature AI-assisted real-time feedback that can dynamically adjust pacing parameters.

### SUMMARY OF THE DISCLOSURE

An esophageal pacing apparatus for use in regulating the heartbeat is disclosed. The apparatus generally comprises an elongated flexible shaft having a proximal end located outside of the esophagus and a distal end located in the esophagus near to the heart. A pacing module is located on the outside of the shaft which measures heartbeat parameters and delivers regulating energy to the heart.

In one embodiment of the present disclosure, the shaft is made flexible by use of a spring element encased in a polymeric outer covering. The outer covering is constructed of a material such as a tetrafluoroethylene polymer (e.g., Teflon®) or can be coated with a hydrophilic material that minimizes friction and eases passage into the esophagus. The shaft includes a plurality of lumens, such as co-axially positioned tubes or multi-lumen tubes, wherein one of the lumens contains a stiffening element having a tapered section at its distal end. The stiffening element allows for accurate control and guidance through the nasal passage into the esophagus. The tapered section of the stiffening wire corresponds to the distal section of the shaft. The tapered section of the stiffening wire is more flexible than the other sections of the wire. This allows for ease of insertion of the distal end of the shaft into a nasal passage or down the throat. Other lumens within the shaft allow for the passage of fluids and various wires used for electrical conductance.

An elongated pacing module is located on the outside of the shaft near its distal end. The pacing module is shaped to increase surface contact with the wall of the esophagus. For example, the pacing module can have an oblong cross section. The pacing module can assume a variety of configurations. For example, the module can be divided into a plurality of oblong sections separated by concave regions. These regions allow the esophagus to collapse therein and increase the contact between the module and esophageal wall.

The pacing module has at least one pacing element, typically a stainless-steel band. This element can both impart electric energy into the wall of the esophagus near to the heart or receive signals from the heart that are used for diagnostic purposes. The esophagus is a muscular tube that extends from the pharynx to the stomach. The esophagus lies posterior to (behind) the trachea and the heart passing through the mediastinum (a central compartment in the chest), and the esophageal hiatus (an opening in the diaphragm where the esophagus enters the abdomen). The lower part of the esophagus lies near to the heart, particularly equal to or less than 10 mm, 9 mm, 8 mm, 7 mm, 6 mm, or 5 mm between the esophagus and the heart (See Sanchez-Quintana et al. (2005) Circulation 112(10): 1400-1405).

The pacing module can also be constructed from a conductive polymer that acts as a sensor and an energy pulsator, thereby eliminating the need for the stainless-steel band. The elongated pacing module also includes an insulated section located on a side opposite side of the pacing element to prevent electrical contact with a non-targeted section of the esophageal wall.

In one embodiment of the present disclosure at least three electrodes are provided. The electrodes are a planar or sinusoidal in nature metal electrode affixed to the side of the oblong pacing module that faces the heart. A first electrode can be tasked with delivering regulating energy pulses to the heart. A second electrode can be utilized as a ground electrode. A third electrode measures an electrocardiogram (EKG) signal to aid in catheter positioning and pacing optimization. For example, the data received from the third electrode can be communicated to as computer utilizing means such as artificial intelligence to determine optimal placement of the pacing module and to regulate the pacing parameters.

A source of electricity is located at the proximal end of the shaft and is in communication with the electrodes. In one embodiment of the present disclosure a pulse generator delivers a desired signal. For example, a high-frequency burst pacing signal can be delivered. In addition, a bioelectric modulation can be delivered which treats atrial fibrillation. The pulse generator can also provide ventricular tachycardia pacing signals or low-energy esophageal defibrillation pulses to treat ventricular fibrillation.

The pulse generator can operate as a closed-loop feedback system, dynamically adjusting energy levels based on the measured EKG and/or hemodynamic data. Cross functionality can be regulated by software or a manual switch located at the proximal end of the shaft allowing selective connection to an energy source or a monitoring system.

### BRIEF DESCRIPTION OF DRAWINGS

The above and other objects, features and advantages of the present disclosure will be more apparent from the following detailed description taken in conjunction with the accompanying drawings, in which:

FIG. 1 is a top cutaway view of a cylindrical catheter with a corresponding pacing element mounted on the catheter that is placed in an esophagus.

FIG. 2 is a top cutaway view of a cylindrical catheter with an elliptical pacing element mounted thereon showing the catheter placed in an esophagus.

FIG. 3 is a side cutaway view of the distal end of the pacing and defibrillation apparatus of the present disclosure.

FIG. 3A is a side cutaway view of the proximal end of the pacing and defibrillation apparatus of the present disclosure.

FIG. 4 is s side cutaway view of the proximal end of the pacing and defibrillation apparatus of the present disclosure.

FIG. 5 is detailed side view showing an electrode mounted on an insulated catheter.

FIG. 6. is a side view of a pacing module of the pacing and defibrillation apparatus of the present disclosure.

FIG. 6A is a cross-section view along line 6A-6A of FIG. 6.

FIG. 7 is a side view of an alternate embodiment of the pacing and defibrillation apparatus of the present disclosure.

FIG. 7A is a cross-section view taken along line 7A-7A of FIG. 7 FIG. 8 is a side view of an alternate embodiment of the pacing and defibrillation apparatus of the present disclosure.

FIG. 9. is a side view of the distal end of an alternate embodiment of the pacing and defibrillation apparatus of the present disclosure.

FIG. 9A. is a cross-section view of the three lumen catheter shaft.

FIG. 10 is a schematic view of showing the pacing and defibrillation apparatus of the present disclosure placed within the esophagus of a patient.

### DETAILED DESCRIPTION OF THE DISCLOSURE

Exemplary embodiments are described herein to provide a detailed description of the present disclosure. Variations of these embodiments will be apparent to those of skill in the art. Moreover, certain terminology is used in the following description for convenience only and is not limiting. For example, the words “right,” “left,” “top,” “bottom,” “upper,” “lower,” “inner” and “outer” designate directions in the drawings to which reference is made. The word “a” is defined to mean “at least one.” The terminology includes the words above specifically mentioned, derivatives thereof, and words of similar importance.

FIG. 1 shows a cross section of the esophagus (100) carrying a cylindrical pacing catheter (110) having a cylindrical pacing electrode. As shown in the diagram, the area of contact with the esophagus is relatively small when compared to total surface area of the cylindrical electrode. The portion marked “X” is the approximate circumference of the pacing electrode that is in contact with the esophageal tissue, i.e. contact length. The present disclosure increases this length thereby providing a greater contact surface of the pacing electrodes that transfer the current into the heart through the esophagus. The catheter shaft in the present disclosure is around 3-6 French (0.04″-0.8″) and allows sufficient space for the introduction of a Nasogastric tube as well.

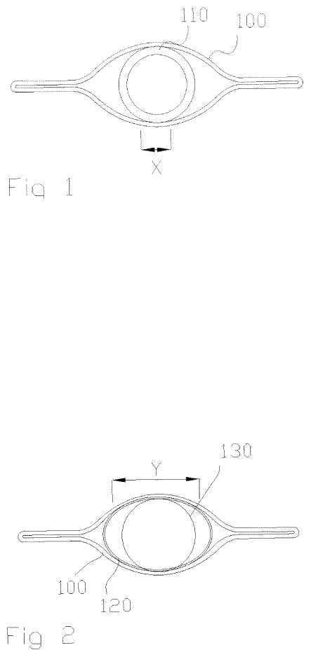

FIG. 2 illustrates the esophagus (100) carrying the pacing and defibrillation module (120) of the present disclosure. Apparatus (120) is shaped to provide an increased length of contact with the wall of the esophagus and can take a variety of shapes that accomplish this. As shown in FIG. 2 the pacing apparatus (120) includes a section that has an oblong or elliptical cross section and provides additional length of contact Y. This length of contact provides for optimal transfer of a pacing pulse to the wall of the esophagus (100). In addition, the pacing apparatus (120) is soft and deformable allowing it to further conform to the esophageal anatomy. As shown in FIG. 3, the catheter shaft (140) is provided with the expanded portion (170) with an oblong or elliptical cross section (170), which carries pacing elements such as bands of electrodes (180). Alternatively, the pacing apparatus (120) can be constructed, in whole or in part, from electrically conductive material. As the area of contact is larger, it will deliver more energy without the need to necessarily increase the magnitude of the pacing pulse.

As shown in FIG. 3 the pacing and defibrillation apparatus (120) of the present disclosure comprises an elongated shaft (140). Shaft (140) comprises a stainless-steel, spring like wrapping (150) having a polymeric outer covering (160). The elongated and oblong pacing module (170) is attached to the shaft (140) near to its distal end (220). The pacing module (170) is shown to carry a plurality of pacing bands, i.e. electrodes (180). Electrically conducting wires (190) attach the pacing bands (180) to the stainless-steel coil shaft to provide the electrical connection from a pulse generator (520), illustrated schematically in FIG. 10, to the pacing bands (180).

A stiffener wire (200) is provided within a lumen of shaft (140) to provide the necessary stiffness to the catheter shaft (140) to improve the guidance and navigation of the catheter (140) through the nostrils (12) or through the mouth (14) of the patient (10) as in FIG. 10. The distal end of the stiffener wire (200) is tapered to provide flexibility to the catheter tip portion at the distal end (220) of the catheter. The stiffener wire has a certain degree of stiffness to maintain the catheter's shape during advancement through the nostrils, mouth, and/or esophagus of the patient, but still flexible enough to bend when required.

The distal end (220) of the catheter is made from soft polymers such as 80A or similar shore hardness Polyurethane or PEBAX type material and is attached to the stainless-steel coil (150) by heat fusing or adhesive bonding. The distal end (220) of the catheter shaft (140) exhibits a preset curvature pointing in the opposite direction of the side having the exposed pacing bands or electrodes (180) to ease insertion through the nostrils (12) or mouth (14). This aspect will save time during the emergent cases requiring patient (10) to be cannulated and paced in an expedited manner.

The proximal end of the apparatus (120), shown in FIG. 3A includes an electrical clamp (230) to connect the stainless-steel coil or spring (150) to the pulse generator (520) via an electrically conducting wire (250), to an insulated hub (260) and a connecting pin (270). The proximal end of the apparatus (120) is provided with a luer lock connector (240) which is attached to the stiffening wire (200). The wire (200) is electrically insulated using a thin a tetrafluoroethylene polymer sheath (210) such that the stiffener wire (200) can be used when the apparatus (120) functions as a bipolar pacing catheter, in which case an additional connector (not shown) can be attached to the stiffener wire (200).

As described in greater detail below, the present disclosure provides that at least one electrode or band (180) is used for grounding while the other band or bands (180) function to provide a pacing and or for defibrillating pulse when in contact with the esophagus (100). Thus, the pacing module serves as a bipolar pacing apparatus. This combination can be used with or without the pad electrode normally used in the patient's chest wall. In addition, the apparatus (120) has at least one electrode or band (180) that is dedicated to obtaining an electrocardiogram signal (EKG) which can be used to ensure optimal positioning of the pacing module (170) proximal to the heart (500).

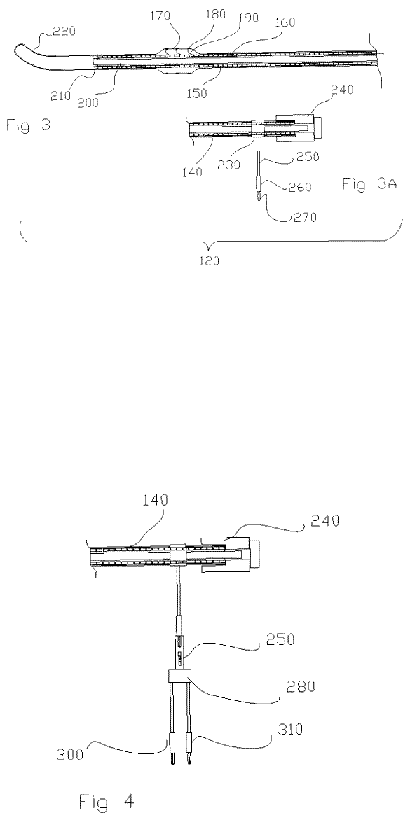

The electrode or band (180) for obtaining the EKG signal can also serve as the pacing electrode by switching the electrode's function at switch (290) as shown in FIG. 4. To use the same electrode to receive the EKG signal as well as for pacing, the pacing electrodes are connected to the switch (290) at the proximal end of the catheter shaft (140). When the switch is in one position it can be connected to the EKG machine (510) and in the flip position it is connected to the pacing or defibrillation pulse generator (520). As shown in FIG. 4, the proximal end of the pacing and defibrillation apparatus (120) is connected to a “Y” hub (280) where a switch (290) is provided to selectively connect the electrical connection wire (250). For example, pacing bands (180) via coil (150) and wire (250) can be coupled with an EKG signal monitor (510). The data from this can then be read and analyzed by a computer (530) that accesses machine learning and artificial intelligence or similar analysis tools. This can indicate accurate positioning, dictate the strength of the pacing or defibrillation pulse delivered, evaluate the efficacy of such pulse and adjust and send further pulses in response.

FIG. 5 is a sectional view of the pacing module (170) which generally has an elliptical shape and includes pacing electrode (180) mounted thereon. The pacing electrode (180) is mounted so as to face the heart while the opposite side has insulation material (320). The insulation (320) prevents the pacing signals from passing into the non-targeted esophageal tissue. This is desirable to avoid any injury during pacing. The insulating material can be either molded or adhesive bonded on to the pacing module (170) in a reliable manner to prevent it getting peeled off during use.

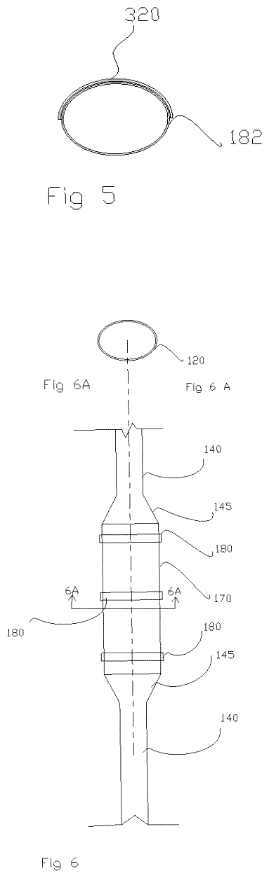

FIG. 6 is a diagrammatic view of the pacing module (170) attached at the distal end of the catheter shaft (140) having tapered ends (145) at both ends. Tapered ends (145) facilitate the entry and exit of the pacing module (170) of catheter shaft (140) into and out of the esophagus (100). The pacing module of FIG. 6 includes a plurality of pacing electrodes (180). As shown in FIG. 6A the cross section of the pacing module (170) is elliptical and the major axis of the ellipse shape of the pacing module is larger than the diameter of the catheter shaft (140) by a multiple of 1.5 to 3 times.

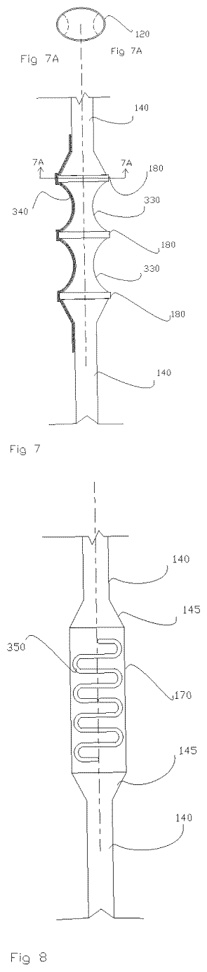

FIG. 7 shows an alternate embodiment of the pacing module (170) where each electrode is interspaced by a concave section (330). This configuration allows the esophageal tissue (340) to bulge into the pacing module (shown only on one side) to have a better contact with the pacing electrode and improve the anchoring of the pacing module in the esophagus. FIG. 7A illustrates a cross-sectional view of this configuration.

Yet another embodiment of the pacing and defibrillation apparatus (120) is shown in FIG. 8. This embodiment utilizes a thin and flexible metal strip (350) attached to the pacing module (170) that provides a superior contact with the esophageal tissue while also proving with an increased distributed area and length along the pacing module (170). This configuration can facilitate and improve the capture of the electrical signal during pacing as well as when used in defibrillation. The electrode is installed only on the pacing side of the pacing module and can be molded into place in a soft plastic such as silicone, soft Polyurethane or Nylon PEBAX type material.

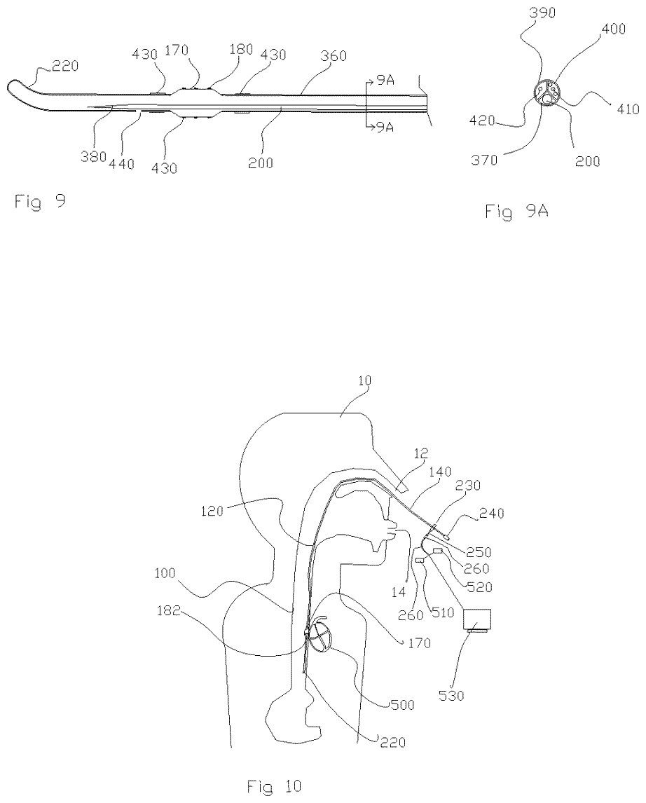

FIG. 9 shows yet another embodiment of the present disclosure. The catheter shaft is a multi-lumen polymeric tube (360), where one lumen (370) houses stiffener wire (200) which increases column strength to facilitate guidance and maneuvering of the tube (360). As described in detail above, the stiffener wire (200) includes a tapered distal portion, shown as pointed tip portion (380). This tapering allows for flexibility at the distal end of the tube (360). As shown in FIG. 9A, lumen (390) houses grounding wires (420) while lumen (400) houses pacing wires (410). The pacing electrodes (180) in the oblong module (170) and the grounding electrodes (430) can be positioned either in the oblong module (170) or somewhere along the catheter shaft (140) relatively close to the pacing module (170). The pacing electrodes (180) and the grounding electrodes (430) can be made from conductive polymer as mentioned earlier and both modules can also be oblong in nature.

As shown in FIG. 9, the catheter shaft (360) includes at least one opening (440). Although shown distal to the pacing module (170) there can be multiple openings (440) at any desired point along the catheter shaft (360). The opening(s) (440) functions to allow for the infusion of medications and fluids to the esophageal wall (100) near the pacing location.

Therapeutics or lubricating fluids can be secreted through the opening (440) or a fluid for maintaining the normal temperature of the esophagus (100) to prevent or minimize any damage caused by any heat generated during pacing. The above-mentioned lumen (440) is in fluid communication with a source located at the proximal end of the shaft (360).

As shown in FIG. (10) the pacing energy delivered by the apparatus (120) is provided by an energy source i.e. pulse generator (510), that delivers energy based on real time data received from the electrodes or pacing bands (180) or pacing module (170) when constructed form an electrically conductive material.

The pulse generator (510) receives the data, for example EKG. The data is analyzed by computer (530) or an analytic system that is onboard pulse generator (510). A user interface is utilized that provides healthcare professionals with analysis results and recommendations, while continuous monitoring adjusts parameters as needed. Pulse generator (510) can provide a high-frequency burst pacing or targeted bioelectric modulation instead of high-energy shocks to treat condition of atrial fibrillation. For a condition such as Ventricular Tachycardia (Vtach) the pulses from the pulse generator (510) can bypass the AV node to directly influence ventricular rhythm. For Ventricular Fibrillation (Vfib) a low-energy esophageal defibrillation pulse at an energy level of (10-30 J) can be combined with pacing. This will produce a less disruptive and painful defibrillation.

Analyzing the EKG waves for pacing or defibrillation commences with preprocessing of the EKG signal which includes noise reduction, baseline correction, R-peak detection, and segmentation into individual heartbeats. Feature extraction identifies critical parameters like R-R intervals, wave durations, and morphological characteristics. Artificial intelligence and machine learning models such as Convolutional Neural Networks (CNNs), Recurrent Neural Networks (RNNs), or Support Vector Machines (SVMs), are trained on labeled EKG datasets to classify various arrhythmia and detect anomalies. A decision-making module, incorporating clinical guidelines or reinforcement learning, then determines appropriate interventions like pacing or defibrillation.

The operation of the esophageal cardiac pacing and defibrillation apparatus (120) is best understood with reference to FIG. 10. The flexible, curved distal end (220) of catheter shaft (140) is inserted into a nasal passage (12) of patient (10). The shaft (140) is advanced to a point in esophagus (100) until the oblong pacing module (170) is proximal to heart (500). The switch (290) is actuated by the end user such that one electrode or band (180) is coupled to an apparatus for measuring and displaying various signals from the heart, such as EKG monitor (520). The signals are analyzed by computer (530) allowing for the pacing module (170) and this data is fed back to the user to ensure optimal placement of module (170).

Upon proper placement of module (170), EKG is again measured and passed back to the end user. Switch (290) is actuated to connect the at least one electrode of band (180) to a pulse generator (510) which supplies a pacing or defibrillation energy pulse to the heart via electrode of band (180). The EKG is then measured again and analyzed wherein the pacing energy to the heart is adjusted accordingly based on the continuously monitored EKG data. The pulse generator (510) can deliver a high-frequency burst pacing for atrial fibrillation, direct ventricular pacing for ventricular tachycardia, or low-energy defibrillation for ventricular fibrillation, as determined by analysis of the EKG data. In the present invention the modules (510), (520) and (530) can all be housed in one unit for convenience for the user.

### Definitions

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

It will be appreciated that various aspects of the invention may be embodied as a method, system, computer readable medium, and/or computer program product. Aspects of the invention may take the form of hardware embodiments, software embodiments (including firmware, resident software, micro-code, etc.), or embodiments combining software and hardware aspects that may all generally be referred to herein as a “circuit,” “module,” or “system.” Furthermore, the methods of the invention may take the form of a computer program product on a computer-usable storage medium having computer-usable program code embodied in the medium.

Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. The computer readable medium may include transitory and/or non-transitory embodiments. More specific embodiments (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Program code for carrying out operations of the invention may be written in an object-oriented programming language such as Java, Smalltalk, C++ or the like. However, the program code for carrying out operations of the invention may also be written in conventional procedural programming languages, such as the “C” programming language or similar programming languages. The program code may be executed by a processor, application specific integrated circuit (ASIC), or other component that executes the program code. The program code may be simply referred to as a software application that is stored in memory (such as the computer readable medium discussed above). The program code may cause the processor (or any processor-controlled device) to produce a graphical user interface (“GUI”). The graphical user interface may be visually produced on a display device, yet the graphical user interface may also have audible features. The program code, however, may operate in any processor-controlled device, such as a computer, server, personal digital assistant, phone, television, or any processor-controlled device utilizing the processor and/or a digital signal processor.

The program code may locally and/or remotely execute. The program code, for example, may be entirely or partially stored in local memory of the processor-controlled device. The program code, however, may also be at least partially remotely stored, accessed, and downloaded to the processor-controlled device. A user's computer, for example, may entirely execute the program code or only partly execute the program code. The program code may be a stand-alone software package that is at least partly on the user's computer and/or partly executed on a remote computer or entirely on a remote computer or server. In the latter scenario, the remote computer may be connected to the user's computer through a communications network.

Certain aspects of invention are described with reference to various methods and method steps. It will be understood that each method step can be implemented by the program code and/or by machine instructions. The program code and/or the machine instructions may create means for implementing the functions/acts specified in the methods.

The program code may also be stored in a computer-readable memory that can direct the processor, computer, or other programmable data processing apparatus to function in a particular manner, such that the program code stored in the computer-readable memory produce or transform an article of manufacture including instruction means which implement various aspects of the method steps.

The program code may also be loaded onto a computer or other programmable data processing apparatus to cause a series of operational steps to be performed to produce a processor/computer implemented process such that the program code provides steps for implementing various functions/acts specified in the methods of the invention.

Although the foregoing subject matter has been described in some detail by way of illustration and example for purposes of clarity of understanding, it will be understood by those skilled in the art that certain changes and modifications can be practiced within the scope of the appended claims.

1. A pacing and defibrillation apparatus for insertion into an esophagus of a patient comprising:
a catheter shaft encased in a stainless-steel spring coil covered with an electrically insulating outer sleeve and having a distal tapered end with a flexible polymeric tip;
a pushable, relatively stiff stainless-steel wire having a coating thereon and contained within a first lumen of the catheter shaft, wherein a distal section of the stainless-steel wire corresponds to a distal section of the catheter shaft, is tapered, and is more flexible than other sections of the stainless-steel wire;
an elongated pacing module, said pacing module having at least two electrodes mounted thereon on a side of the pacing module that is adapted to be proximal to the heart;
a pulse generator configured to receive an electrocardiogram (EKG) signal from one of the at least two electrodes; and
a computer configured to:
preprocess the EKG signal by noise reduction, baseline correction, R-peak detection, and segmentation into individual heartbeats;
extract R-R intervals, wave durations, and morphological characteristics from the EKG signal;
use at least one artificial intelligence or machine learning model selected from a Convolutional Neural Network (CNN), a Recurrent Neural Network (RNN), or a Support Vector Machine (SVM), the model being trained on labeled EKG datasets to classify cardiac arrhythmias and detect anomalies; and
use a decision-making module incorporating clinical guidelines or reinforcement learning to determine an appropriate intervention comprising pacing or defibrillation;
wherein based on the determined intervention, the pulse generator generates an electrical pulse providing pacing or defibrillation of the heart.

2. The apparatus of claim 1, wherein the pulse generator delivers:
a low-energy esophageal defibrillation pulse at an energy level of 10-30 J combined with pacing to treat ventricular fibrillation.

3. The apparatus of claim 1, wherein the pulse generator operates as a closed-loop feedback system, dynamically adjusting energy levels based on an EKG and a hemodynamic data.

4. The apparatus of claim 1, wherein the elongated pacing module has an elliptical cross section with a major axis that is 1.5 to 3 times a diameter of the catheter shaft and includes a plurality of oblong sections, adjacent oblong sections being separated by a concave region, wherein an electrode is mounted on a heart-facing side of each oblong section and an opposite side of the elongated pacing module has insulation material that prevents pacing signals from passing into non-targeted esophageal tissue.

5. The apparatus of claim 1, wherein the catheter shaft comprises a second lumen with wires for connecting at least one of the at least two electrodes to the pulse generator.

6. The apparatus of claim 5, wherein the catheter comprises a third lumen in fluid communication with at least one opening located distal to the elongated pacing module, the third lumen being configured to transmit a fluid through the at least one opening to the esophageal wall near a pacing location for maintaining a normal temperature of the esophagus to prevent or minimize damage caused by heat generated during pacing.

7. The apparatus of claim 1, wherein the distal tapered end exhibits a preset curvature pointing in a direction opposite the side of the elongated pacing module having the at least two electrodes.
